(19) United States
(12) Patent Application Publication
(10) Pub. No.: US 2021/0054342 A1
(43) Pub. Date: Feb. 25, 2021

(54) MESENCHYMAL STEM CELLS EXPRESSING CD146 RECEPTORS

(71) Applicant: Riam Shammaa, Toronto (CA)

(72) Inventors: Riam Shammaa, Toronto (CA); Rafei Moutih, Montreal (CA)

(21) Appl. No.: 16/996,786

(22) Filed: Aug. 18, 2020

Related U.S. Application Data
(60) Provisional application No. 62/889,438, filed on Aug. 20, 2019.

(51) Int. Cl. C12N 5/0775

(52) U.S. Cl. CPC C12N 5/0662

(57) ABSTRACT

A composition comprising MSCs wherein said MSCs express CD146 receptors in an amount of at least 10 pg/10⁶ cells.

### CROSS-REFERENCE TO RELATED APPLICATION

This application claims the benefit under 35 U.S.C. § 119 of U.S. Provisional Patent Application No. 62/889,438, filed on Aug. 20, 2019, which is incorporated herein by reference in its entirety.

### FIELD OF INVENTION

The present invention relates to mesenchymal stem cells (MSCs), and in particular, to MSCs expressing CD146 receptors. These MSCs inhibit the proliferation of lymphocytes and may be employed, for example, in the treatment of osteoarthritis, degenerative disc disease, Crohn's and inflammatory bowel disease, multiple sclerosis, and graft-versus-host disease.

### BACKGROUND OF THE INVENTION

MSCs are multipotent stem cells that can differentiate readily into lineages including osteoblasts, myocytes, chondrocytes, and adipocytes (Pittenger, et al., Science. Vol. 284, pg. 143 (1999); Haynesworth, et al., Bone. Vol. 13, pg. 69 (1992); Prockop, Science, Vol. 276, pg. 71 (1997)). In vitro studies have demonstrated the capability of MSCs to differentiate into muscle (Wakitani, et al., Muscle Nerve. Vol. 18, pg. 1417 (1995)), neuronal-like precursors (Woodbury, et al., J. Neurosci. Res., Vol. 69, pg. 908 (2002); Sanchez-Ramos, et al., Exp. Neurol., Vol. 171, pg. 109 (2001)), cardiomyocytes (Toma, et al., Circulation. Vol. 105, pg. 93 (2002); Fukuda, Artif. Organs. Vol. 25, pg. 187 (2001)) and possibly other cell types. In addition, MSCs have been shown to provide effective feeder layers for expansion of hematopoietic stem cells (Wagers, et al., Gene Therapy. Vol. 9, pg. 606 (2002)).

Recent studies with a variety of animal models have shown that MSCs may be useful in the repair or regeneration of damaged bone, cartilage, meniscus or myocardial tissues (DeKok, et al., Clin. Oral Implants Res., Vol. 14, pg. 481 (2003)); Wu, et al., Transplantation. Vol. 75, pg. 679 (2003); Mackenzie, et al., Blood Cells Mol. Pis., Vol. 27, pgs. 601-604 (2001)). Several investigators have used MSCs with encouraging results for transplantation in animal disease models including osteogenesis imperfecta (Pereira, et al., Proc. Nat. Acad. Sci., Vol. 95, pg. 1142 (1998)), parkinsonism, spinal cord injury and cardiac disorders (Tomita, et al., Circulation. Vol. 100, pg. 247 (1999). Shake, et al., Ann. Thorac. Sura., Vol. 73, pg. 1919 (2002)). Importantly, promising results also have been reported in clinical trials for osteogenesis imperfecta and enhanced engraftment of heterologous bone marrow transplants.

In addition, in vitro studies from different laboratories have shown that MSCs can inhibit T-cell proliferation either in mixed lymphocyte cultures or by other stimuli such as antigens and mitogens (Di Nicola, et al., Blood, Vol. 99, pgs. 3638-3843 (2002); Tse, et al., Transplantation. Vol. 75, pgs. 389-397 (2003); Aggarwal, et al., Blood, Vol. 105, pgs. 1815-1822 (2005)). Recent in vitro data demonstrate further that MSCs decrease the secretion of pro-inflammatory cytokines, tumor necrosis factor-a (TNF-a), and Interferon-y (IFN-y), and simultaneously increase production of anti-inflammatory cytokines Interleukin-10 (IL-10) and Interleukin-4 (IL-4) by immune cells. (Aggarwal, 2005). These results indicate that due to immunomodulatory and anti-inflammatory activities, MSCs can be beneficial for treatment of immunological responses which occur in graft-versus-host disease (GVHD), solid organ transplantation, and autoimmune diseases such as multiple sclerosis and rheumatoid arthritis. A clinical case report demonstrating the therapeutic effect of MSCs for acute GVHD supports strongly this hypothesis. (Le Blanc, et al., The Lancet, Vol. 363, pgs. 1439-1441 (2004).)

It would be advantageous to provide MSCs that inhibit the proliferation of lymphocytes and may be employed, for example, in the treatment of osteoarthritis, degenerative disc disease and inflammatory disease.

CD146 (cluster of differentiation 146), also known as the melanoma cell adhesion molecule (MCAM) and cell surface glycoprotein MUC18, is a 113 kDa cell adhesion molecule used as a marker for endothelial cell lineage. In humans, the CD146 protein is encoded by the MCAM gene.

CD10 (cluster of differentiation 10), also known as neprilysin, membrane metallo-endopeptidase (MME), neutral endopeptidase (NEP), and common acute lymphoblastic leukemia antigen (CALLA), is an enzyme that in humans is encoded by the MME gene. CD10 is a zinc-dependent metalloprotease that cleaves peptides at the amino side of hydrophobic residues and inactivates several peptide hormones. It also degrades the amyloid beta peptide whose abnormal folding and aggregation in neural tissue has been implicated as a cause of Alzheimer's disease. Synthesized as a membrane-bound protein, the neprilysin ectodomain is released into the extracellular domain after it has been transported from the Golgi apparatus to the cell surface.

### SUMMARY

In one aspect, the present invention provides a composition comprising mesenchymal stem cells, wherein said mesenchymal stem cells express CD146 receptors in an amount of at least 10 pg/10⁶ cells.

In another aspect, the present invention provides a method of obtaining MSCs which express CD146 receptors in an amount of at least 10 pg/10⁶ cells, comprising: obtaining at least one cell population including mesenchymal stem cells from at least one donor; determining the amount of CD146 expressed by the mesenchymal stem cells in each of said at least one cell population(s); and selecting mesenchymal stem cells which express CD146 receptors in an amount of at least 10 pg/10⁶ cells.

Additional features and benefits of the present invention will become apparent from the detailed description, figures and claims set forth below.

### BRIEF DESCRIPTION OF THE DRAWINGS

The exemplary embodiments of the present invention will be understood more fully from the detailed description given below and from the accompanying drawings of various embodiments of the invention, which, however, should not be taken to limit the invention to the specific embodiments, but are for explanation and understanding only.

FIGS. 1A-1C provide graphs showing the phenotypic characterization of CD146+ MSCs: (A) Flow-cytometry analysis of CD146^{lo} versus CD146^{hi} MSCs following their sorting from the parental MSC population; (B) Comparative analysis of cell proliferation over time, with the parental MSC population displayed at the top, and CD146^{lo} MSCs and CD146^{hi} MSCs below; (C) Phenotypic analysis of the three different MSC populations according to the ISCT guidelines, with isotype controls are shown as filled/shaded histograms and the vertical dotted line is placed according to the CD146^{hi} MSC population.

FIG. 2 provides graphs showing analysis of immune related molecules. Isotype controls are shown as filled/shaded histograms. The vertical dotted line is placed according to the CD146^{hi} MSC population.

FIG. 3 shows the results of the secreatome analysis. Luminex analysis of various cytokines and chemokines in the conditioned media derived from the parental MSC population, CD146^{lo} MSC or CD146^{hi} MSC. For this experiment, n=6/group with *P<0.05 and ***P<0.01.

FIG. 4 provides a graph showing CXCL12 quantification by ELISA. Parental MSCs, CD146^{lo} MSCs and CD146^{hi} MSCs were used to quantify levels of CXCL12 using a commercial ELISA. For this experiment, n=5/group with *P<0.05 and ***P<0.01.

### DETAILED DESCRIPTION OF PREFERRED EMBODIMENTS

The present invention relates to MSCs. More particularly, this invention relates to mesenchymal stem cells which express CD146 receptors, and in particular, express CD146 receptors in an amount of at least 10 pg/10⁶ cells. Such MSCs secrete high levels of CCL2 (MCP-1) and CXCL12. CCL2 and CXCL12 can cooperatively polarize macrophages to secrete IL-10, which can suppress immune responses. As such, the present invention provides a subpopulation of “Super Suppressor Cells”.

In accordance with a preferred aspect of the present invention, there is provided a composition comprising mesenchymal stem cells. The MSCs express the CD146 receptors in an amount effective to inhibit immune responses.

In one embodiment, the MSCs express CD146 receptors in an amount of at least 10 pg/10⁶ cells. In another embodiment, the MSCs express CD146 receptors in an amount of at least 12 pg/10⁶ cells, more preferably at least 15 pg/10⁶ cells, and even more preferably at least 18 pg/10⁶ cells.

The inventor has found that MSCs which express CD146 receptors in an amount of at least 10 pg/10⁶ cells may be useful in inhibiting immune responses, and more particularly, such MSCs may be useful in the treatment osteoarthritis, degenerative disc disease, and autoimmune diseases such as, for example, lyme disease, rheumatoid arthritis, multiple sclerosis, Type I diabetes, Crohn's disease, graft-versus-host disease, Guillain-Barre syndrome, lupus erythematosus, myasthenia gravis, optic neuritis, psoriasis, Graves' disease, Hashimoto's disease, Ord's thyroiditis, aplastic anemia, Reiter's syndrome, autoimmune hepatitis, primary biliary cirrhosis, antiphospholipid antibody syndrome, opsoclonus myoclonus syndrome, temporal arteritis, acute disseminated encephalomyelitis, Goodpasture's syndrome, Wegener's granulomatosis, coeliac disease, pemphigus, polyarthritis, warm autoimmune hemolytic anemia, and scleroderma.

In another preferred embodiment, the MSCs express CD10 receptors in an amount of at least 10 pg/10⁶ cells. More preferably, the MSCs express CD10 receptors in an amount of at least 12 pg/10⁶ cells, even more preferably in an amount of at least 15 pg/10⁶ cells, and most preferably in an amount of at least 18 pg/10⁶ cells.

In one preferred embodiment, the MSCs are obtained from a mammal. The mammal may be a primate, including human and non-human primates.

The MSCs may be a homogeneous composition or may be a mixed cell population enriched in MSCs. Homogeneous mesenchymal stem cell compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium.

The amount of cellular CD146 receptors that is expressed in a culture of MSCs may be determined by methods known to those skilled in the art. Such methods include, but are not limited to, quantitative assays such as quantitative ELISA assays, for example. It is to be understood, however, that the scope of the present invention is not to be limited to any particular method for determining the amount of CD146 receptors.

In one embodiment, the amount of CD146 receptors expressed by a culture of MSCs is determined by an ELISA assay. In such an assay, a cell lysate from a culture of MSCs is added to a well of an ELISA plate. The well may be coated with an antibody, either a monoclonal or a polyclonal antibody(ies), against the CD146 receptors. The well then is washed, and then contacted with an antibody, either a monoclonal or a polyclonal antibody(ies), against the CD146 receptors. The antibody is conjugated to an appropriate enzyme, such as horseradish peroxidase, for example. The well then may be incubated, and then is washed after the incubation period. The wells then are contacted with an appropriate substrate, such as one or more chromogens. Chromogens which may be employed include, but are not limited to, hydrogen peroxide and tetramethylbenzidine. After the substrate(s) is (are) added, the well is incubated for an appropriate period of time.

Upon completion of the incubation, a “stop” solution is added to the well in order to stop the reaction of the enzyme with the substrate(s). The optical density (OD) of the sample then is measured. The optical density of the sample is correlated to the optical densities of samples containing known amounts of CD146 receptors in order to determine the amount of CD146 expressed by the culture of MSCs being tested.

Thus, the present invention provides for the selection of a population of MSCs which express CD146 receptors in an amount of at least 10 pg/10⁶ cells. Such selected MSCs then may be admixed with an appropriate pharmaceutical carrier for treatment of the diseases and disorders mentioned hereinabove. For example, the MSCs may be administered as a cell suspension including a pharmaceutically acceptable liquid medium for injection.

The MSCs of the present invention are administered to an animal in an amount effective to treat one or more of the above-mentioned diseases or disorders in the animal. The animal may be a mammal, and the mammal may be a primate, including human and non-human primates. The MSCs may be administered systemically, such as, for example, by intravenous, intra-arterial, or intraperitoneal administration. The exact dosage of MSCs to be administered is dependent upon a variety of factors, including, but not limited to, the age, weight, and sex of the patient, the disease(s) or disorder(s) being treated, and the extent and severity thereof.

The expression level of CD146 on hMSCs correlates with hMSC immunosuppressive activity. The level of CD146 expression by hMSCs of less than 10 pg/10⁶ cells has been found to be a threshold, below which hMSCs may begin to lose their ability to suppress an immune response. Thus, CD146 expression is a marker of hMSC immunosuppression, an activity that is believed essential for MSCs to be efficacious for treatment of immunological reactions taking place in osteoarthritis, degenerative disc disease, autoimmune diseases, and other diseases.

### Experimental Section

### Animals and Ethics

All female C57BL/6 and Balb/c mice (6-8 weeks old) were purchased from the Jackson Laboratory (Bar Harbor, Me., USA) and housed in a pathogen-free environment at the animal facility of the Institute for Research in Immunology and Cancer (IRIC). All experimental procedures and protocols were approved by the Animal Ethics Committee of Université de Montreal.

### Antibodies and Reagents

All flow-cytometry antibodies were purchased from BD Biosciences (San Jose, Calif., USA). All ELISA kits were purchased from R&D Systems (Minneapolis, Minn., USA).

### Generation of BM-Derived MSCs

In order to generate MSCs, femurs of 6-8 weeks old female C57BL/6 mice were isolated and flushed with Alpha Modification of Eagle's Medium (AMEM) supplemented with 10% FBS, and 50 U/mL Penicillin-Streptomycin in a 10 cm cell culture dish (CellStar), then incubated at 37° C. Two days later, non-adherent cells were removed and the media was replaced every 3 to 4 days until the cells reached 80% confluency. Adherent cells were detached using 0.05% trypsin and expanded until a uniform MSC population is obtained. Generated MSCs were validated by flow-cytometry for the expression of CD44, CD45, CD73, and CD105.

### MSC Sorting, Phenotypic Analysis and Cell Counting

To isolate CD146^{lo} versus CD146^{hi} MSCs, the parental population was stained with anti-CD146 antibodies and the 5% low or high CD146 positive cells were sorted. Following their culture, the generated MSC populations were stained with various antibodies directed against immune molecules (MHCI, CD80, CD86 and PD-L1). A study was also conducted to evaluate their proliferation rate. For this purpose, 5×103 MSCs were plated in a 6 well plate and cell numbers counted every 24 hrs. Parental MSCs were used as control.

### Cytokine and Chemokine Analysis

For cytokine and chemokine profiling, 15 cm culture petri dishes containing 80-90% confluent MSC were grown in serum-free media for 24 hr at 37° C. Collected supernatants were then analyzed using luminex by Eve Technologies (Calgary, AB, CA).

### Results

### MSC Characterization

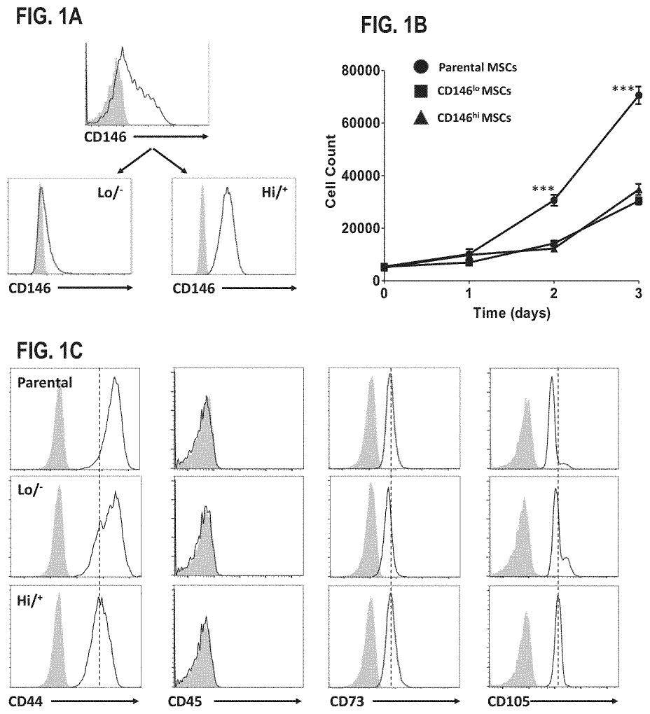

Following MSC sorting, a flow-cytometry assessment of CD146 was conducted on MSCs (see FIG. 1A). To confirm that the cells do not exhibit idiosyncratic characteristic, a cell proliferation analysis was conducted. Although no differences could be depicted between the CD146^{lo} and CD146^{hi} MSC populations, their proliferation rate was substantially lower than that of the parental population (see FIG. 1B). Phenotypic analysis of the different MSC populations, on the other hand, revealed the fact that CD146^{hi} MSCs express lower levels of CD44 but exhibits higher CD105 expression (see FIG. 1C). These results indicate that the CD146 marker can lead to a phenotypic difference within a heterogeneous MSC population.

### Immune Profiling of MSCs

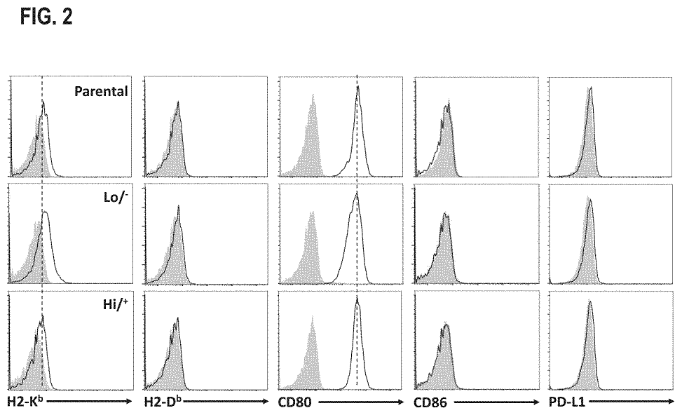

Based on the previous difference observed in the phenotype of MSCs, we next analyzed various immune-related molecules by flow-cytometry. The three MSC populations were compared for their expression profile of MHCI (H2-Kb and H2-Db), costimulatory molecules (CD80 and CD86) as well as for the immune checkpoint blocker. Although most markers were within the same range, a low but detectable difference was observed for H2-Kb (MHCI molecules) whereas CD80 expression was slightly higher on the surface of CD146^{hi} MSCs. Results are shown in FIG. 2.

CD146 Depicts MSC Populations with Different Secreatome Profiles

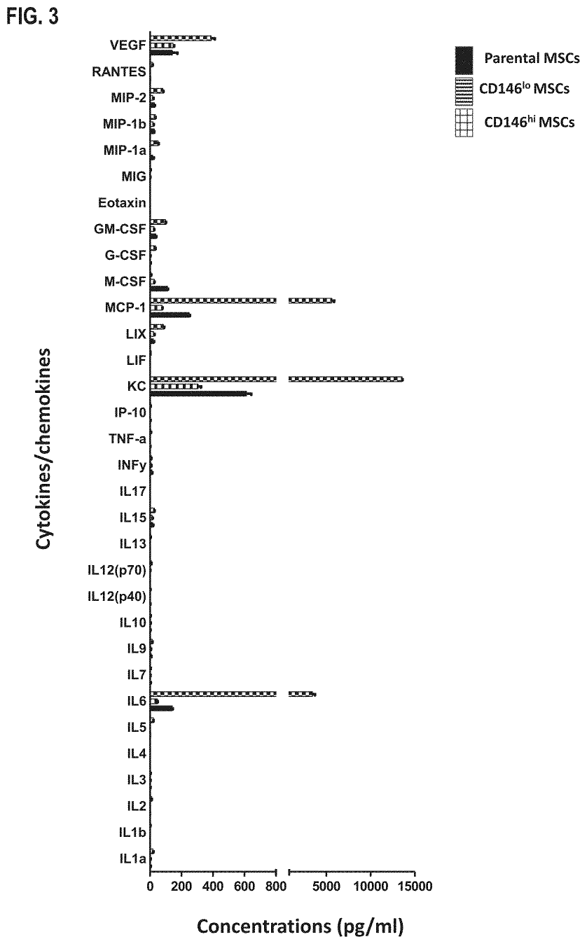

To study the effect of segregating MSCs based on CD146 expression, the supernatant derived from the parental MSC population was compared to supernatants derived from CD146^{lo} or CD146^{hi} MSCs. Secreatome analysis revealed substantial differences between the different populations with IL-6, KC, MCP-1 and VEGF being highly secreted by MSCs expressing high CD146 levels. Besides, MSC-CD146^{hi} produced also significantly higher amounts of LIX, G-CSF, GM-CSF, MIP-1alpha and MIP-2, which are all potent chemoattractant for different immune subtypes. Overall, these data clearly show that separating MSCs based on their CD146 expression leads to variables differences in their secreatome profile. Results are shown in FIG. 3.

CXCL12 Quantification by ELISA

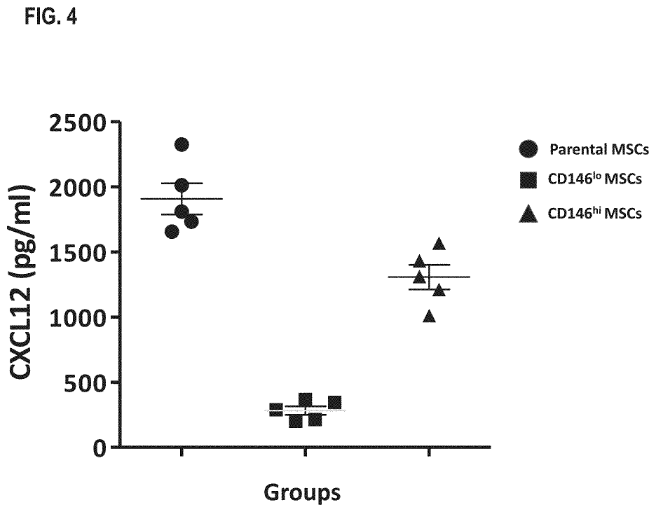

Parental MSCs, CD146^{lo} MSCs and CD146^{hi} MSCs were cultured overnight in serum-free media. Collected supernatants were then used to quantify the levels of CXCL12 using a commercial ELISA. For this experiment, n=5/group with *P<0.05 and ***P<0.01. Results are shown in FIG. 4. CD146hi MSCs secreted high levels of CXCL12.

It is to be understood, however, that the scope of the present invention is not to be limited to the specific embodiments described above. The invention may be practiced other than as particularly described and still be within the scope of the accompanying claims.

What is claimed is:

1. A composition comprising mesenchymal stem cells wherein said mesenchymal stem cells express CD146 receptors in an amount of at least 10 pg/10⁶ cells.

2. The composition of claim 1, wherein said MSCs express CD146 receptors in an amount of at least 12 pg/10⁶ cells.

3. The composition of claim 2, wherein said mesenchymal stem cells express CD146 receptors in an amount of at least 15 pg/10⁶ cells.

4. The composition of claim 3, wherein said mesenchymal stem cells express CD146 receptors in an amount of at least 18 pg/10⁶ cells.

5. The composition of claim 1, wherein said mesenchymal stem cells are human mesenchymal stem cells.

6. The composition of claim 1, further comprising an acceptable pharmaceutical carrier.

7. A method of obtaining mesenchymal stem cells which express CD146 receptors in an amount of at least 10 pg/10⁶ cells, comprising:
obtaining at least one cell population including mesenchymal stem cells from at least one donor;
determining the amount of CD146 expressed by the mesenchymal stem cells in each of said at least one cell population; and
selecting mesenchymal stem cells which express CD146 receptors in an amount of at least 10 pg/10⁶ cells.

8. The method of claim 7, wherein said selected mesenchymal stem cells express CD146 receptors in an amount of at least 12 pg/10⁶ cells.

9. The method of claim 8, wherein said selected mesenchymal stem cells express CD146 receptors in an amount of at least 15 pg/10⁶ cells.

10. The method of claim 9, wherein said selected mesenchymal stem cells express CD146 receptors in an amount of at least 18 pg/10⁶ cells.

11. The method of claim 7, wherein said mesenchymal stem cells are human mesenchymal stem cells.
